(19) United States
(12) Patent Application Publication
(10) Pub. No.: US 2023/0310377 A1
(43) Pub. Date: Oct. 5, 2023

(54) METHODS OF TREATING LYSOSOMAL STORAGE DISEASES AND METHODS RELATED THERETO

(71) Applicant: RECURSION PHARMACEUTICALS, INC., SALT LAKE CITY, UT (US)

(72) Inventors: YOLANDA CHONG, SALT LAKE CITY, UT (US); TRAVIS RUSH, SALT LAKE CITY, UT (US); MICHAEL STATNICK, SALT LAKE CITY, UT (US); DANIEL ANDERSON, SALT LAKE CITY, UT (US); MICHAEL GENIN, SALT LAKE CITY, UT (US)

(73) Assignee: RECURSION PHARMACEUTICALS, INC., SALT LAKE CITY, UT (US)

(21) Appl. No.: 18/007,048

(22) PCT Filed: Jul. 28, 2021
(86) PCT No.: PCT/US2021/043528

§ 371 Date: Jan. 27, 2023

Related U.S. Application Data
(60) Provisional application No. 63/057,803, filed on Jul. 28, 2020.

(51) Int. Cl. A61K 31/407; A61K 31/517; A61K 31/4545; A61K 31/5517; A61K 31/5377; A61K 31/497; A61P 25/28

(52) U.S. Cl. CPC A61K 31/407; A61K 31/517; A61K 31/4545; A61K 31/5517; A61K 31/5377; A61K 31/497; A61P 25/28

(57) ABSTRACT

Methods and compositions are provided for treating lysosomal storage diseases and symptoms thereof. The methods include administering a PKC inhibitor, a GSK3β inhibitor, or an inhibitor of both.

### CROSS-REFERENCE TO RELATED APPLICATIONS

This application claims priority to U.S. provisional patent application No. 63/057,803, filed Jul. 28, 2020 and entitled “Treating GM2 Gangliosidosi,” the contents of which are incorporated herein in their entirety.

### TECHNICAL FIELD

The present disclosure relates to methods of treating diseases. More particularly, the disclosure relates to methods of treating lysosomal storage diseases (LSDs) and methods related thereto.

### BACKGROUND

Lysosomal storage diseases (LSDs) are inherited metabolic disorders that are distinguished by an abnormal build-up of various materials in the body's cells as a result of defects in lysosomal function. Lysosomes are vesicles that contain enzymes that can break down many kinds of molecules. When there is an enzyme deficiency, then materials build up in cells. The accumulation of these materials causes a variety of problems, which can vary based on the type of material. New LSDs continue to be discovered. There continues to be a need for development of treatments for LSDs and associated symptoms.

### BRIEF DESCRIPTION OF THE DRAWINGS

The embodiments disclosed herein will become more fully apparent from the following description and appended claims, taken in conjunction with the accompanying drawings.

FIG. 1 depicts the images of immunofluorescence-labeled cells used in experiments disclosed herein, including after administration of 1.1 microMolar ruboxistaurin.

FIG. 2A-1 depicts the dose response with ruboxistaurin.

FIG. 2A-2 depicts the dose response with sotrastaurin.

FIG. 2A-3 depicts the dose response with enzastaurin.

FIG. 2B-1 depicts mean fluorescence or mean aggregate count with ruboxistaurin.

FIG. 2B-2 depicts mean fluorescence or mean aggregate count with sotrastaurin.

FIG. 2B-3 depicts mean fluorescence or mean aggregate count with enzastaurin.

FIG. 2C depicts nuclei per image with ruboxistaurin.

FIG. 2D-1 depicts mean cytoplasmic autofluorescence with ruboxistaurin.

FIG. 2D-2 depicts mean cytoplasmic autofluorescence with sotrastaurin.

FIG. 3A depicts the dose response from another experiment with ruboxistaurin.

FIG. 3B depicts mean fluorescence or mean aggregate count for the other experiment with ruboxistaurin.

FIG. 3C depicts nuclei per image from the other experiment with ruboxistaurin.

FIG. 4A-1 depicts the dose response for LY2090314 from a first experiment (Expmt 1936).

FIG. 4A-2 depicts the dose response for LY2090314 from a second experiment (Expmt 1925).

FIG. 4B-1 depicts the mean aggregate count from Expmt 1936 for LY2090314.

FIG. 4B-2 depicts the nuclei count per image from Expmt 1936 for LY2090314.

FIG. 4B-3 depicts the mean aggregate count from Expmt 1925 for LY2090314.

FIG. 4B-4 depicts the nuclei count per image from Expmts 1925 for LY2090314.

FIG. 5A-1 depicts the dose response for AZD1080 from a first experiment (Expmt 1936).

FIG. 5A-2 depicts the dose response for AZD1080 from a second experiment (Expmt 1925).

FIG. 5B-1 depicts the mean aggregate count from Expmt 1936 for AZD1080.

FIG. 5B-2 depicts the nuclei count per image from Expmt 1936 for AZD1080.

FIG. 5B-3 depicts the mean aggregate count from Expmt 1925 for AZD1080.

FIG. 5B-4 depicts the nuclei count per image from Expmts 1925 for AZD1080.

FIG. 6A-1 depicts the dose response for AZD2858 from a first experiment (Expmt 1936).

FIG. 6A-2 depicts the dose response for AZD2858 from a second experiment (Expmt 1925).

FIG. 6B-1 depicts the mean aggregate count from Expmt 1936 for AZD2858.

FIG. 6B-2 depicts the nuclei count per image from Expmt 1936 for AZD2858.

FIG. 6B-3 depicts the mean aggregate count from Expmt 1925 for AZD2858.

FIG. 6B-4 depicts the nuclei count per image from Expmts 1925 for AZD2858.

### DETAILED DESCRIPTION

The present disclosure relates to methods of treating diseases. More particularly, the disclosure relates to methods of treating lysosomal storage diseases (LSDs) and methods related thereto. It will be readily understood that the embodiments, as generally described herein, are exemplary. The following more detailed description of various embodiments is not intended to limit the scope of the present disclosure but is merely representative of various embodiments. Moreover, the order of the steps or actions of the methods disclosed herein may be changed by those skilled in the art without departing from the scope of the present disclosure. In other words, unless a specific order of steps or actions is required for proper operation of the embodiment, the order or use of specific steps or actions may be modified.

A first aspect of the disclosure relates to methods of treating lysosomal storage diseases and symptoms thereof (and relatedly compositions for use in treating lysosomal storage diseases and symptoms thereof and methods of manufacturing compositions for treating lysosomal storage diseases and symptoms thereof). The methods comprise administering a therapeutically effective amount of a compound comprising a PKC inhibitor, a GSK3β inhibitor, or an inhibitor of both to a subject in need of treatment. For example, the compound may comprise ruboxistaurin, sotrastaurin, enzastaurin, LY2090314, AZD1080, AZD2858, prodrugs, active metabolites, analogs, or derivatives of the foregoing or a pharmaceutically acceptable salt, solvate, or ester of the foregoing. The methods may include administering salts of ruboxistaurin such as hydrochloride, sulfate, mesylate, succinate, tartrate, acetate, or phosphate salts. Particular solvates may also be administered, such as ruboxistaurin mesylate monohydrate. The methods may include administering an active metabolite of ruboxistaurin, such as the major metabolite N-desmethyl ruboxistaurin. The methods may include administering an analog of ruboxistaurin, such as (S)-10,11,14,15-tetrahydro-13-hydroxymethyl-4,9,16,21-dimetheno-1H,13H-dibenzo[e,k]pyrrolo[3,4-h][1,4,13]oxadiazacyclohexadecene-1,3(2H)-dione (referred to herein as “Compound A”).

The chemical structure of ruboxistaurin is:

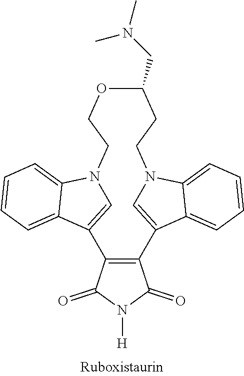 [SMILES: CN(C)C[C@@H]1CCn2cc(c3ccccc32)C2=C(C(=O)NC2=O)c2cn(c3ccccc23)CCO1]

The IUPAC name for ruboxistaurin is (18S)-18-[(dimethylamino)methyl]-17-oxa-4,14,21-triazahexacyclo[19.6.1.17,14.02,6.08,13.022,27]nonacosa-1(28),2(6),7(29),8,10,12,22,24,26-nonaene-3,5-dione. The CAS number for ruboxistaurin is 169939-94-0. Ruboxistaurin is also known as LY333531. Ruboxistaurin mesylate is also known as Arxxant. Ruboxistaurin is both a PKC inhibitor and a GSK3β inhibitor. Ruboxistaurin has an experimentally determined GM2 ganglioside ED₅₀ of ˜300 nM and autofluorescence ED₅₀ of ˜250 nM.

A major metabolite of ruboxistaurin is known as N-desmethyl ruboxistaurin. The chemical structure of N-desmethyl ruboxistaurin is:

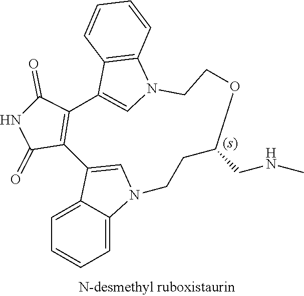 [SMILES: CNC[C@@H]1CCn2cc(c3ccccc32)C2=C(C(=O)NC2=O)c2cn(c3ccccc23)CCO1]

N-desmethyl ruboxistaurin is also known as LY338522. The CAS number for N-desmethyl ruboxistaurin is 191848-32-5. The name according to Scifinder is (9S)-6,7,10,11-Tetrahydro-9-[(methylamino)methyl]-9H,18H-5,21:12,17-dimethenodibenzo[e,k]pyrrolo[3,4-h][1,4,13]oxadiazacyclohexadecine-18,20(19H)-dione.

Compound A has a chemical structure of:

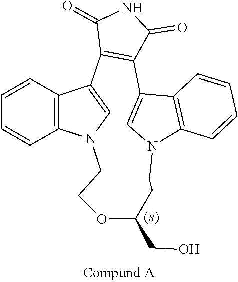 [SMILES: O=C1NC(=O)C2=C1c1cn(c3ccccc13)CCO[C@H](CO)CCn1cc2c2ccccc21]

The CAS number for Compound A is 169940-55-0.

A “ metabolite” of ruboxistaurin is a derivative that is formed when the compound is metabolized. The term “active metabolite” refers to a biologically active derivative of ruboxistaurin that is formed when the compound is metabolized. The term “metabolized,” as used herein, refers to the sum of the processes (including, but not limited to, hydrolysis reactions and reactions catalyzed by enzymes) by which a particular substance is changed by an organism. Thus, enzymes may produce specific structural alterations to a compound. For example, cytochrome P450 catalyzes a variety of oxidative and reductive reactions while uridine diphosphate glucuronyltransferases catalyze the transfer of an activated glucuronic-acid molecule to aromatic alcohols, aliphatic alcohols, carboxylic acids, amines and free sulphydryl groups.

Sotrastaurin and enzastaurin are analogs of ruboxistaurin.

Sotrastaurin has a chemical structure of:

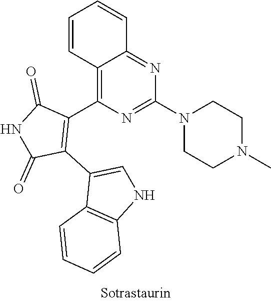 [SMILES: CN1CCN(c2nc(C3=C(c4c[nH]c5ccccc45)C(=O)NC3=O)c3ccccc3n2)CC1]

The CAS number for Sotrastaurin is 425637-18-9. Sotrastaurin is a PKC inhibitor and has an experimentally determined GM2 ganglioside ED₅₀ of ˜90 nM.

Enzastaurin has a chemical structure of:

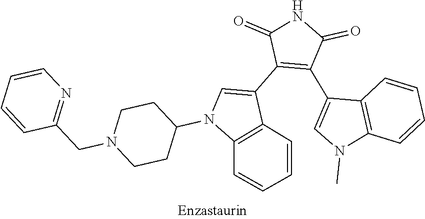 [SMILES: Cn1cc(C2=C(c3cn(C4CCN(Cc5ccccn5)CC4)c4ccccc34)C(=O)NC2=O)c2ccccc21]

The CAS number for Enzastaurin is 170364-57-5. Enzastaurin is a PKC inhibitor and has an experimentally determined GM2 ganglioside ED₅₀ of ˜265 for HEXA and ˜550 nM for HEXB.

LY2090314 has a chemical structure of:

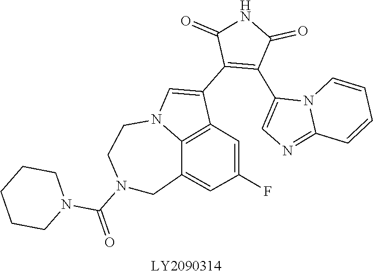 [SMILES: O=C1NC(=O)C(c2cnc3ccccn23)=C1c1cn2c3c(cc(F)cc13)CN(C(=O)N1CCCCC1)CC2]

The CAS number for LY2090314 is 603288-22-8. LY2090314 is a GSK3β inhibitor.

AZD1080 has a chemical structure of:

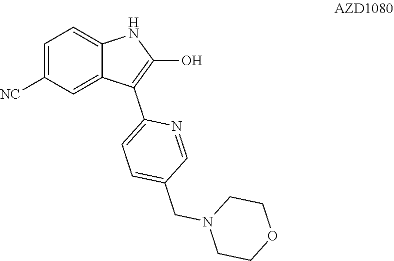 [SMILES: N#Cc1ccc2[nH]c(O)c(-c3ccc(CN4CCOCC4)cn3)c2c1]

The CAS number for AZD1080 is 612487-72-6. AZD1080 is a GSK3β inhibitor.

AZD2858 has a chemical structure of:

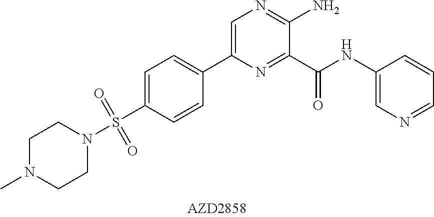 [SMILES: CN1CCN(S(=O)(=O)c2ccc(-c3cnc(N)c(C(=O)Nc4cccnc4)n3)cc2)CC1]

The CAS number for AZD2858 is 486424-20-8. AZD2858 is a GSK3β inhibitor. As used herein, “administering” encompasses either administering a compound, or a pharmaceutical compositions comprising the compound, directly to isolated cells or to an animal, or administering to cells or an animal another agent to cause the presence or formation of the compound inside the cells or the animal. Accordingly, the “another agent” may administered in a sufficient amount to achieve a therapeutically effective amount of the compound inside the cells or the animal.

The salts as mentioned herein are meant to comprise the therapeutically active non-toxic acid addition salt forms which the compounds disclosed herein are able to form. The salts can conveniently be obtained by treating the base form with such appropriate acids as inorganic acids, for example, hydrohalic acids, e.g. hydrochloric, hydrobromic and the like; sulfuric acid; nitric acid; phosphoric acid and the like; or organic acids, for example, acetic, propanoic, hydroxy-acetic, 2-hydroxypropanoic, 2-oxopropanoic, oxalic, malonic, succinic, maleic, fumaric, malic, tartaric, 2-hydroxy-1,2,3-propanetricarboxylic, methanesulfonic, ethanesulfonic, benzenesulfonic, 4-methylbenzenesulfonic, cyclohexanesulfamic, 2-hydroxybenzoic, 4-amino-2-hydroxybenzoic and the like acids. Conversely the salt form can be converted by treatment with alkali into the free base form.

The compounds disclosed herein containing acidic protons may be converted into their therapeutically active non-toxic metal or amine addition salt forms by treatment with appropriate organic and inorganic bases. Appropriate base salt forms comprise, for example, the ammonium salts, the alkali and earth alkaline metal salts, e.g. the lithium, sodium, potassium, magnesium, calcium salts and the like, salts with organic bases, e.g. primary, secondary and tertiary aliphatic and aromatic amines such as methylamine, ethylamine, propylamine, isopropylamine, the four butylamine isomers, dimethylamine, diethylamine, diethanolamine, dipropylamine, diisopropylamine, di-n-butylamine, pyrrolidine, piperidine, morpholine, trimethylamine, triethylamine, tripropylamine, quinuclidine, pyridine, quinoline and isoquinoline, the benzathine, N-methyl-D-glucamine, 2-amino-2-(hydroxymethyl)-1,3-propanedi-ol, hydrabamine salts, and salts with amino acids such as, for example, arginine, lysine and the like. Conversely the salt form can be converted by treatment with acid into the free acid form.

The term solvate includes the hydrates and solvent addition forms which the compounds disclosed herein are able to form. Examples of such forms are e.g. hydrates, alcoholates and the like.

Pharmaceutically acceptable salts of the compounds disclosed herein include all salts that are exemplified by alkaline salts with an inorganic acid and/or a salt with an organic acid that are known in the art. In addition, pharmaceutically acceptable salts include acid salts of inorganic bases, as well as acid salts of organic bases.

Derivatives of the compounds disclosed herein include N-oxide derivatives, quaternary amines, and deuterated derivatives. N-oxide derivatives of the compounds disclosed herein are meant to include compounds where one or several nitrogen atoms are oxidized to the so-called N-oxide. “Quaternary amine” refers to any quaternary ammonium salts which the compounds disclosed herein are able to form by reaction between a basic nitrogen of a compound disclosed herein and an appropriate quaternizing agent, such as, for example, an optionally substituted alkylhalide, arylhalide or arylalkylhalide, e.g. methyliodide or benzyliodide. Other reactants with good leaving groups may also be used, such as alkyl trifluoromethanesulfonates, alkyl methanesulfonates, and alkyl p-toluenesulfonates. A quaternary amine has a positively charged nitrogen. Pharmaceutically acceptable counterions include chloro, bromo, iodo, trifluoroacetate and acetate. The counterion of choice can be introduced using ion exchange resins. Deuterated derivatives refers to compounds disclosed herein where any bound hydrogen atom is substituted with a deuterium atom.

In any of the embodiments disclosed herein, administering a therapeutically effective amount of a compound comprising a PKC inhibitor, a GSK3β inhibitor, or an inhibitor of both may comprise administering a composition or formulation consisting essentially of a PKC inhibitor, a GSK3β inhibitor, or an inhibitor of both; may comprise administering a composition or formulation consisting essentially of a PKC inhibitor; comprises administering a composition or formulation consisting essentially of a GSK3β inhibitor; may comprise administering a composition or formulation consisting essentially of an inhibitor of both PKC and GSK3β; may comprise administering a composition or formulation consisting essentially of ruboxistaurin, prodrugs, active metabolites, analogs, or derivatives thereof, a pharmaceutically acceptable salt, solvate, or ester of the foregoing, or combinations thereof; may comprise administering a composition or formulation consisting essentially of sotrastaurin, prodrugs, active metabolites, analogs, or derivatives thereof, a pharmaceutically acceptable salt, solvate, or ester of the foregoing, or combinations thereof; may comprise administering a composition or formulation consisting essentially of enzastaurin, prodrugs, active metabolites, analogs, or derivatives thereof, a pharmaceutically acceptable salt, solvate, or ester of the foregoing, or combinations thereof; may comprise administering a composition or formulation consisting essentially of LY2090314, prodrugs, active metabolites, analogs, or derivatives thereof, a pharmaceutically acceptable salt, solvate, or ester of the foregoing, or combinations thereof; may comprise administering a composition or formulation consisting essentially of AZD1080, prodrugs, active metabolites, analogs, or derivatives thereof, a pharmaceutically acceptable salt, solvate, or ester of the foregoing, or combinations thereof; or may comprise administering a composition or formulation consisting essentially of AZD2858, prodrugs, active metabolites, analogs, or derivatives thereof, a pharmaceutically acceptable salt, solvate, or ester of the foregoing, or combinations thereof.

In any of the embodiments disclosed herein, the administering may include orally administering. Parenteral routes of administration are also possible, such as intracerebroventricular, intravenous, intramuscular, subcutaneous, and transdermal.

In any of the embodiments disclosed herein, the lysosomal storage disease (LSD) treated may be a sphingolipidosis. In particular, the LSD may be a gangliosidosis. Even more particularly, the LSD may be a GM2 gangliosidosis, such as infantile GM2 gangliosidosis, Tay-Sachs disease or Sandhoff disease.

In any of the embodiments disclosed herein, the subject may be 36 months old or less at the time of starting treatment.

In any of the embodiments disclosed herein, the symptoms may include one or more of: progressive loss of motor skills; retinal abnormalities; myoclonic seizures; increased startle response; severe constipation; loss of visceral organ function; cherry red spot; hypotonia; dyskinesia; and dystonia.

Another aspect of the disclosure relates to methods of treating diseases and symptoms associated with mutation in a hexosaminidase subunit alpha (HEXA) gene of a subject, the method comprising administering a therapeutically effective amount of a compound comprising a PKC inhibitor, a GSK3β inhibitor, or an inhibitor of both to a subject in need of treatment. For example, the disease may be Tay-Sachs disease and the subject may be a mammal. The compound may comprise ruboxistaurin, sotrastaurin, enzastaurin, LY2090314, AZD1080, AZD2858, prodrugs, active metabolites, analogs, or derivatives of the foregoing or a pharmaceutically acceptable salt, solvate, or ester of the foregoing.

The methods may include administering salts of ruboxistaurin such as hydrochloride, sulfate, mesylate, succinate, tartrate, acetate, or phosphate salts. Particular solvates may also be administered, such as ruboxistaurin mesylate monohydrate. The methods may include administering an active metabolite of ruboxistaurin, such as the major metabolite N-desmethyl ruboxistaurin. The methods may include administering an analog of ruboxistaurin, such as (S)-10,11,14,15-tetrahydro-13-hydroxymethyl-4,9,16,21-dimetheno-1H,13H-dibenzo[e,k]pyrrolo[3,4-h][1,4,13]oxadiazacyclohexadecene-1,3(2H)-dione (referred to herein as “Compound A”).

Another aspect of the disclosure relates to methods of treating diseases and symptoms associated with mutation in a hexosaminidase subunit beta (HEXB) gene of a subject, the method comprising administering a therapeutically effective amount of a compound comprising a PKC inhibitor, a GSK3β inhibitor, or an inhibitor of both to a subject in need of treatment. For example, the disease may be Sandhoff disease and the subject may be a mammal. The compound may comprise ruboxistaurin, sotrastaurin, enzastaurin, LY2090314, AZD1080, AZD2858, prodrugs, active metabolites, analogs, or derivatives of the foregoing or a pharmaceutically acceptable salt, solvate, or ester of the foregoing.

The methods may include administering salts of ruboxistaurin such as hydrochloride, sulfate, mesylate, succinate, tartrate, acetate, or phosphate salts. Particular solvates may also be administered, such as ruboxistaurin mesylate monohydrate. The methods may include administering an active metabolite of ruboxistaurin, such as the major metabolite N-desmethyl ruboxistaurin. The methods may include administering an analog of ruboxistaurin, such as (S)-10,11,14,15-tetrahydro-13-hydroxymethyl-4,9,16,21-dimetheno-1H,13H-dibenzo[e,k]pyrrolo[3,4-h][1,4,13]oxadiazacyclohexadecene-1,3(2H)-dione (referred to herein as “Compound A”).

Another aspect of the disclosure relates to methods of treating cells, such as the cells of a mammal, with increased ganglioside accumulation, the method comprising administering an effective amount of a compound comprising a PKC inhibitor, a GSK3β inhibitor, or an inhibitor of both to the cells. When the cells are in or from a mammal, the methods may further include identifying the mammal as having cells with ganglioside accumulation. Identifying the mammal as having cells with ganglioside accumulation may involve obtaining a cell sample from a patient and measuring GM2 fluorescence of the patient's cells.

Administering an effective amount of a compound comprising a PKC inhibitor, a GSK3β inhibitor, or an inhibitor of both may comprise administering a composition or formulation consisting essentially of a PKC inhibitor, a GSK3β inhibitor, or an inhibitor of both; may comprise administering a composition or formulation consisting essentially of a PKC inhibitor; may comprise administering a composition or formulation consisting essentially of a GSK3β inhibitor; may comprise administering a composition or formulation consisting essentially of an inhibitor of both PKC and GSK3β; may comprise administering a composition or formulation consisting essentially of ruboxistaurin, prodrugs, active metabolites, analogs, or derivatives thereof, a pharmaceutically acceptable salt, solvate, or ester of the foregoing, or combinations thereof; may comprise administering a composition or formulation consisting essentially of sotrastaurin, prodrugs, active metabolites, analogs, or derivatives thereof, a pharmaceutically acceptable salt, solvate, or ester of the foregoing, or combinations thereof; may comprise administering a composition or formulation consisting essentially of enzastaurin, prodrugs, active metabolites, analogs, or derivatives thereof, a pharmaceutically acceptable salt, solvate, or ester of the foregoing, or combinations thereof; may comprise administering a composition or formulation consisting essentially of LY2090314, prodrugs, active metabolites, analogs, or derivatives thereof, a pharmaceutically acceptable salt, solvate, or ester of the foregoing, or combinations thereof; may comprise administering a composition or formulation consisting essentially of AZD1080, prodrugs, active metabolites, analogs, or derivatives thereof, a pharmaceutically acceptable salt, solvate, or ester of the foregoing, or combinations thereof; or may comprise administering a composition or formulation consisting essentially of AZD2858, prodrugs, active metabolites, analogs, or derivatives thereof, a pharmaceutically acceptable salt, solvate, or ester of the foregoing, or combinations thereof. The administering may include orally administering.

The methods may include administering salts of ruboxistaurin such as hydrochloride, sulfate, mesylate, succinate, tartrate, acetate, or phosphate salts. Particular solvates may also be administered, such as ruboxistaurin mesylate monohydrate. The methods may include administering an active metabolite of ruboxistaurin, such as the major metabolite N-desmethyl ruboxistaurin. The methods may include administering an analog of ruboxistaurin, such as (S)-10,11,14,15-tetrahydro-13-hydroxymethyl-4,9,16,21-dimetheno-1H,13H-dibenzo[e,k]pyrrolo[3,4-h][1,4,13]oxadiazacyclohexadecene-1,3(2H)-dione (referred to herein as “Compound A”).

### EXAMPLES

To further illustrate these embodiments, the following examples are provided. These examples are not intended to limit the scope of the claimed invention, which should be determined solely on the basis of the attached claims.

### Example 1—Ganglioside Accumulation

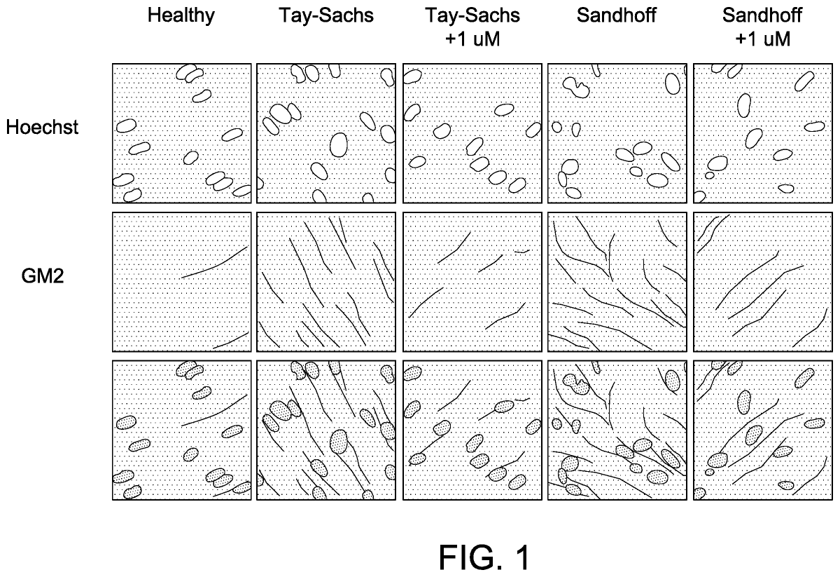

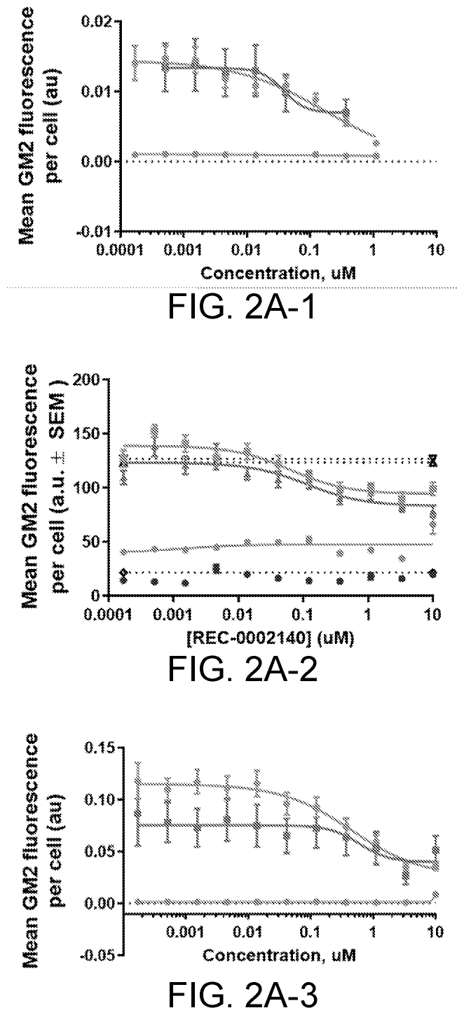

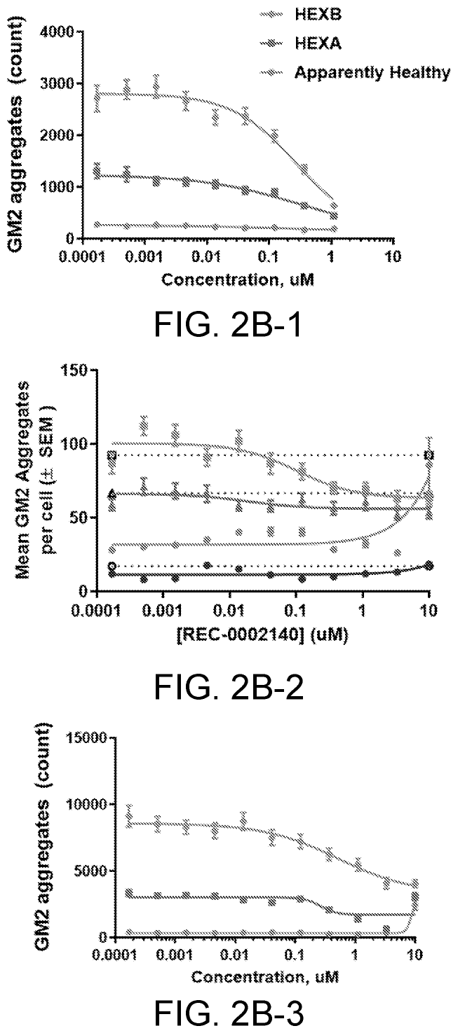

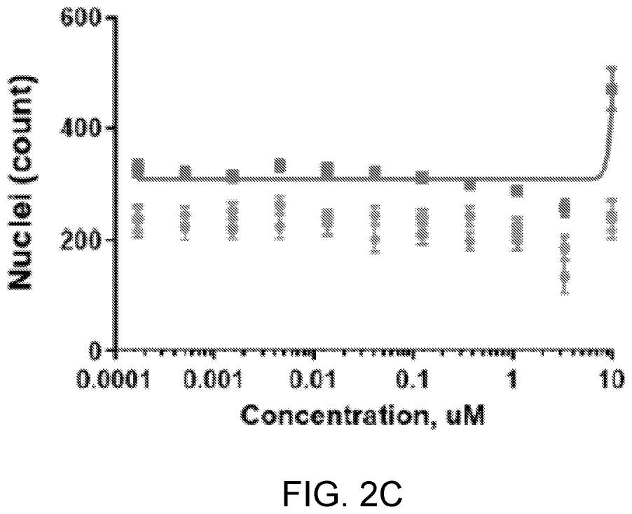

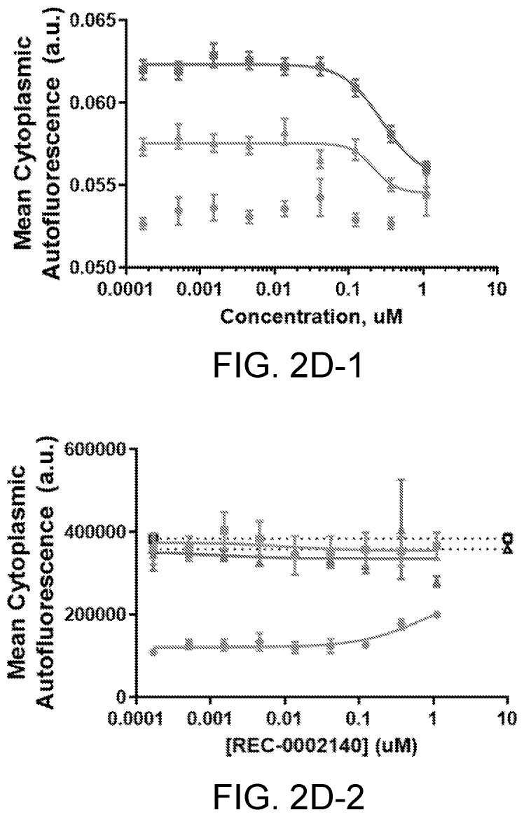

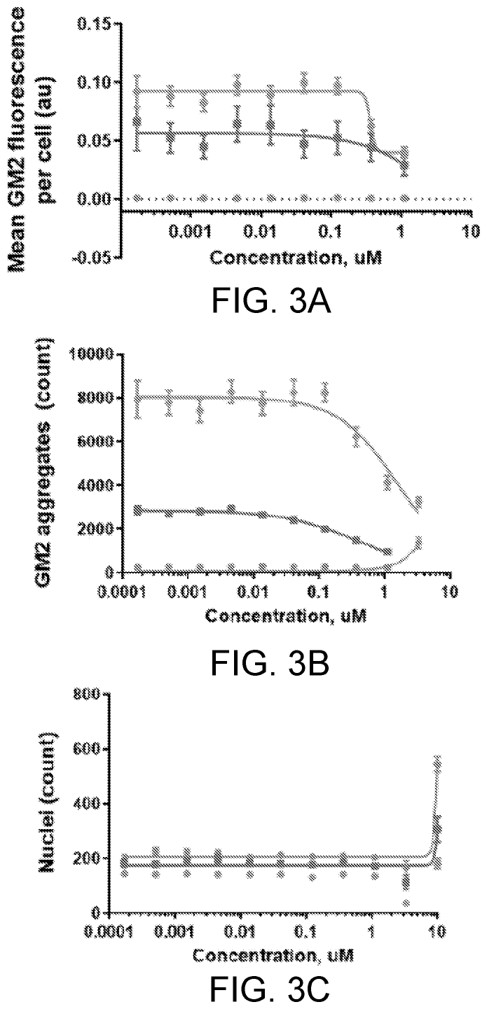

Healthy, Sandhoff (HEXB) disease, and Tay-Sachs (HEXA) disease patient fibroblasts were plated and treated with ruboxistaurin mesylate twice over 17 days. At day 0 cells were seeded. On day 5 the media was changed and initial dose of the compound was delivered. On day 10 the media was changed again and the second dose of the compound was delivered. On day 17 the cells were fixed, immunoflourescence-labeled, and imaged for GM2 (see FIG. 1). Sandhoff and Tay-Sachs disease patient fibroblasts exhibited higher mean GM2 fluorescence and aggregate counts, which were dose-dependently rescued with ruboxistaurin mesylate treatment (ED₅₀=256.3 nM, collective ED₅₀ for the GM2 mean fluorescence rescue across HEXA and HEXB patient lines in the experiments). FIGS. 2A-1 and 3A depict the dose response for two separate experiments. FIGS. 2B-1 and 3B depict mean fluorescence or mean aggregate count for each experiment±SEM of technical replicates, combining 3-5 patient lines for each group. n=12-40 images from two well replicates imaged at 4-sites per well for each line at each dose. Data in each panel represents n=1 experiment performed. FIGS. 2C and 3C depict nuclei per image from the same image sets.

The same experiment was performed for each of sotrastaurin, enzastaurin, LY2090314, AZD1080, and AZD2858. FIG. 2A-2 depicts the dose response for sotrastaurin. FIG. 2A-3 depicts the dose response for enzastaurin.

FIG. 2B-2 depicts mean fluorescence or mean aggregate count for sotrastaurin. FIG. 2B-3 depicts mean fluorescence or mean aggregate count for enzastaurin.

FIGS. 2D-1 and 2D-2 depict mean cytoplasmic autofluorescence for ruboxistaurin and sotrastaurin, respectively.

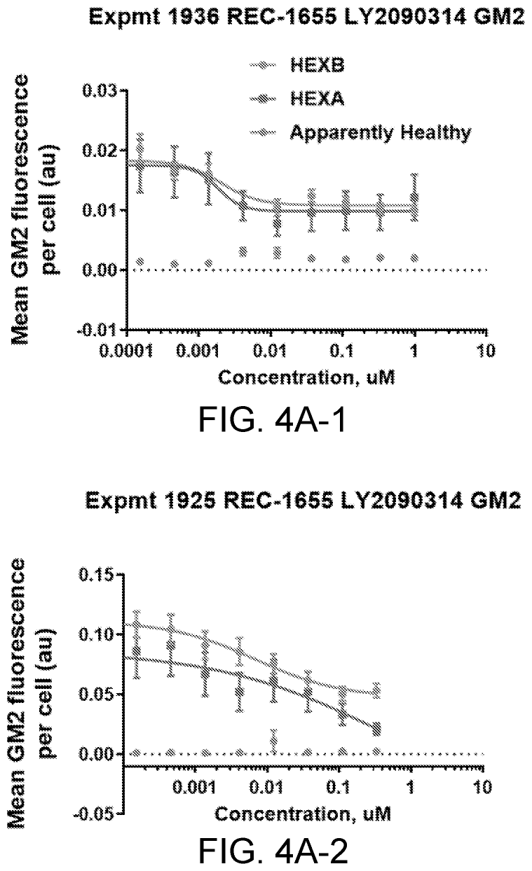

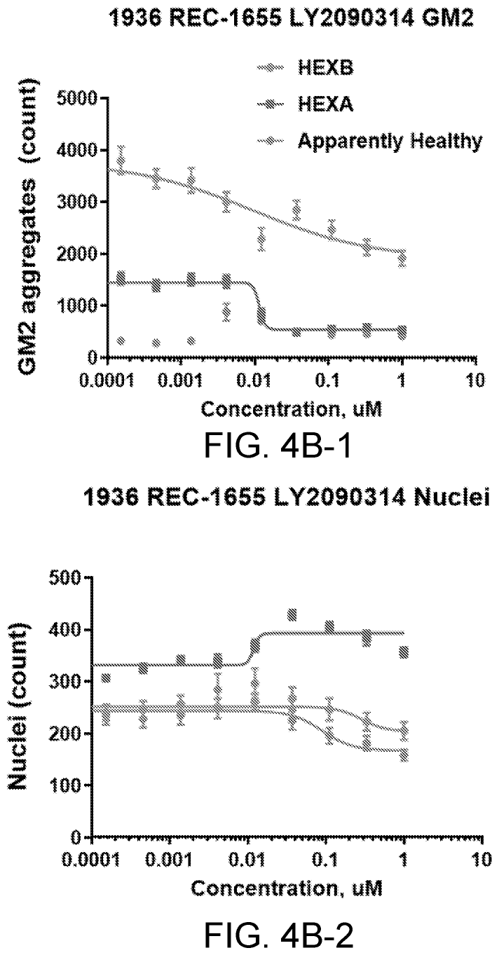

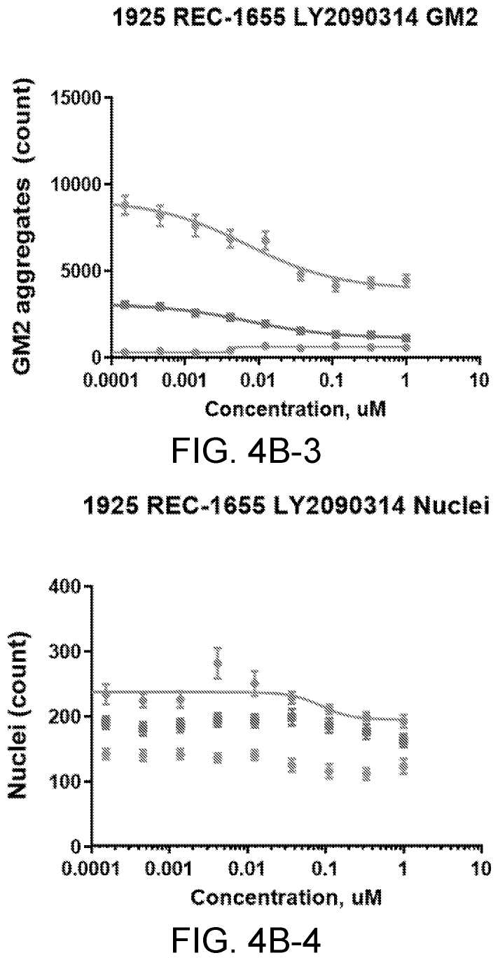

FIGS. 4A-1 and 4A-2 depict the dose response for LY2090314 from two different experiments (Expmt 1936 and 1925, respectively). FIGS. 4B-1 and 4B-3 depict the mean aggregate count from Expmts 1936 and 1925 for LY2090314, respectively. FIGS. 4B-2 and 4B-4 depict the nuclei count per image from Expmts 1936 and 1925 for LY2090314, respectively. The ED₅₀ was 8.76 nM. Experiment 1925 used older/higher-passage number cells. Experiment 1936 used freshly thawed/lower-passage number cells. EC₅₀ calculations were based on GM2 aggregate counts (not mean fluorescence).

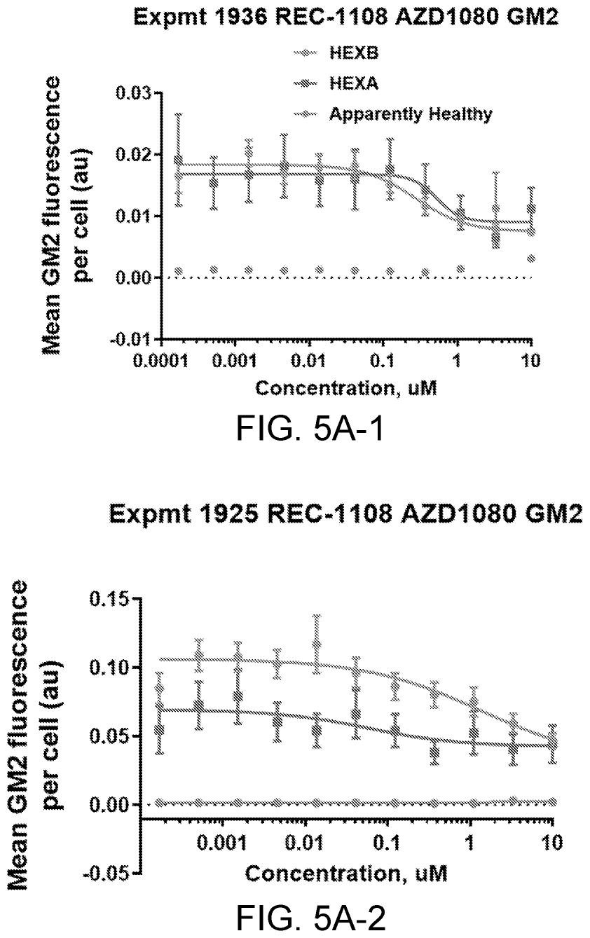

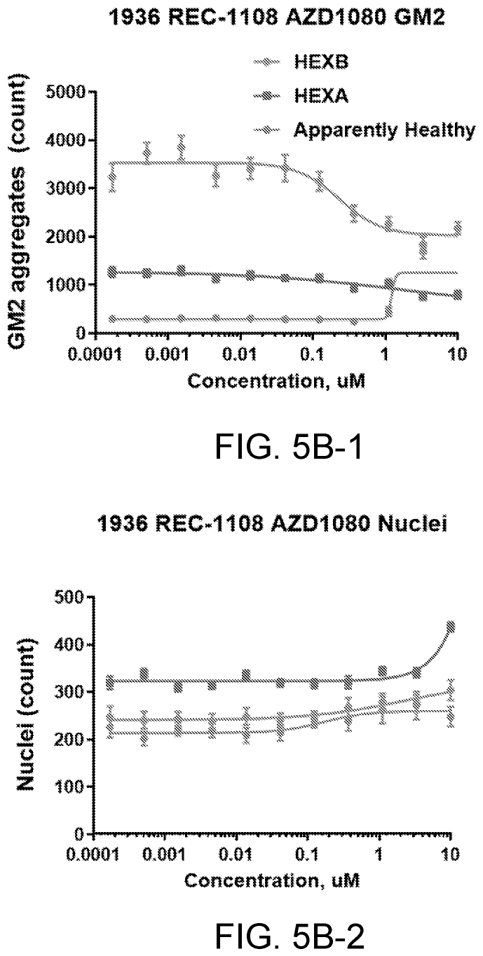

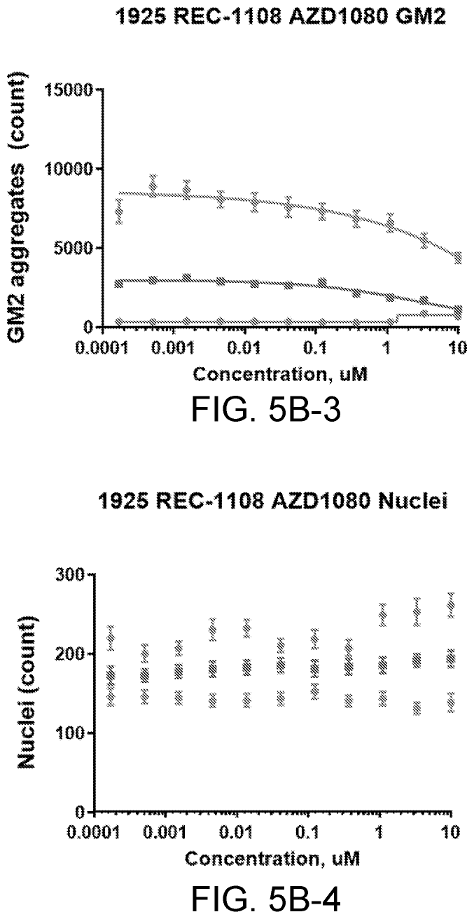

FIGS. 5A-1 and 5A-2 depict the dose response for AZD1080 from two different experiments (Expmt 1936 and 1925, respectively). FIGS. 5B-1 and 5B-3 depict the mean aggregate count from Expmts 1936 and 1925 for AZD1080, respectively. FIGS. 5B-2 and 5B-4 depict the nuclei count per image from Expmts 1936 and 1925 for AZD1080, respectively. The ED₅₀ was 1591.3 nM. Experiment 1925 used older/higher-passage number cells. Experiment 1936 used freshly thawed/lower-passage number cells. EC₅₀ calculations were based on GM2 aggregate counts (not mean fluorescence).

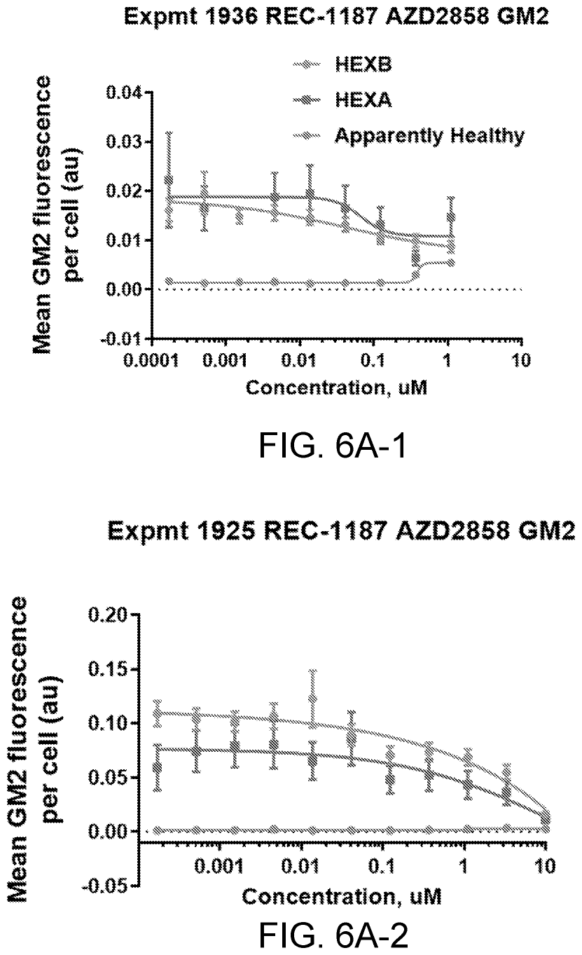

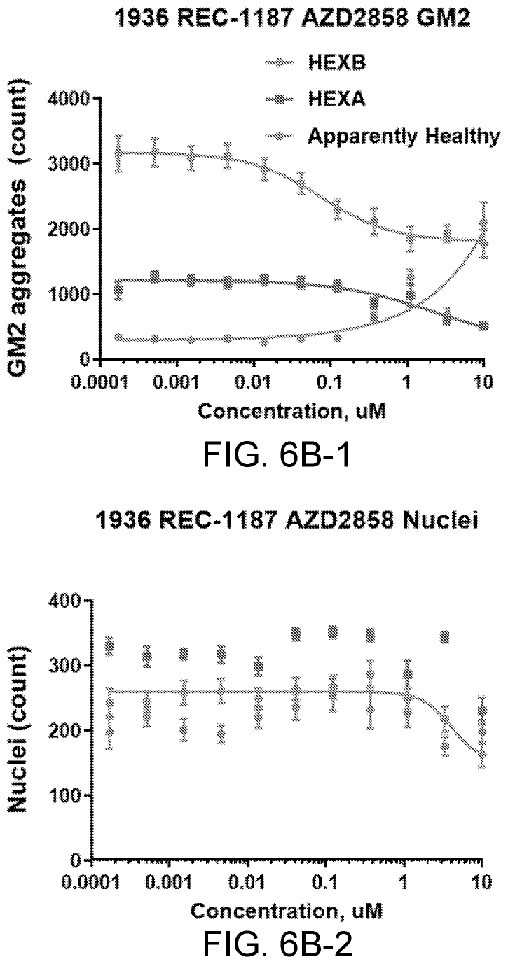

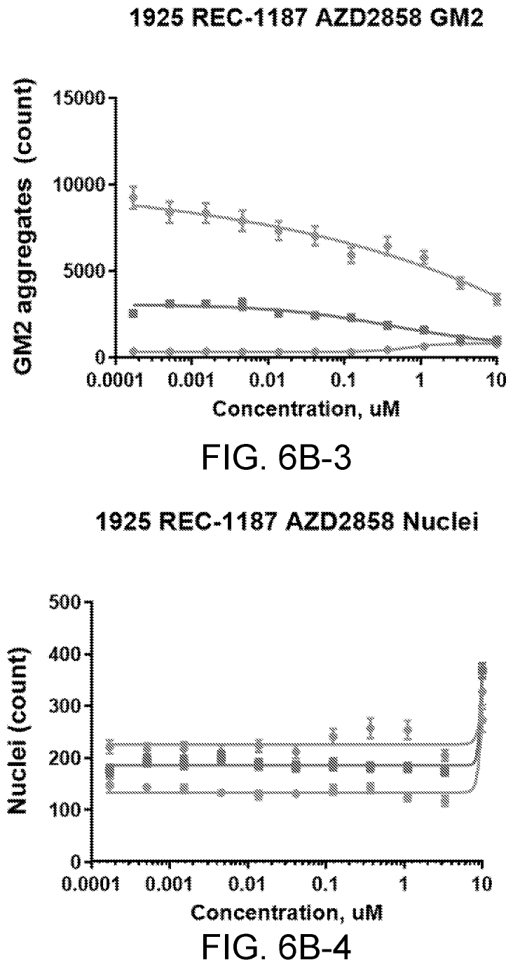

FIGS. 6A-1 and 6A-2 depict the dose response for AZD2858 from two different experiments (Expmt 1936 and 1925, respectively). FIGS. 6B-1 and 6B-3 depict the mean aggregate count from Expmts 1936 and 1925 for AZD2858, respectively. FIGS. 6B-2 and 6B-4 depict the nuclei count per image from Expmts 1936 and 1925 for AZD2858, respectively. The ED₅₀ was 938.0 nM. Experiment 1925 used older/higher-passage number cells. Experiment 1936 used freshly thawed/lower-passage number cells. EC₅₀ calculations were based on GM2 aggregate counts (not mean fluorescence).

The leftmost concentration in all graphs is DMSO control.

It will be apparent to those having skill in the art that many changes may be made to the details of the above-described embodiments without departing from the underlying principles of the invention.

1. A method of treating lysosomal storage diseases and symptoms thereof, the method comprising administering a therapeutically effective amount of a compound comprising a PKC inhibitor, a GSK3β inhibitor, or an inhibitor of both to a subject in need of treatment.

2. The method of claim 1, wherein the compound comprises ruboxistaurin, sotrastaurin, enzastaurin, LY2090314, AZD1080, AZD2858, prodrugs, active metabolites, analogs, or derivatives of the foregoing or a pharmaceutically acceptable salt, solvate, or ester of the foregoing.

3. The method of claim 1, comprising administering ruboxistaurin, prodrugs, active metabolites, analogs, or derivatives thereof, a pharmaceutically acceptable salt, solvate, or ester of the foregoing, or combinations thereof.

4. The method of claim 1, comprising administering ruboxistaurin hydrochloride, sulfate, mesylate, succinate, tartrate, acetate, or phosphate.

5. The method of claim 1, comprising administering ruboxistaurin mesylate monohydrate.

6. The method of claim 1, comprising administering N-desmethyl ruboxistaurin, a pharmaceutically acceptable salt, solvate, or ester thereof, or combinations thereof.

7. The method of claim 1, comprising administering (S)-10,11,14,15-tetrahydro-13-hydroxymethyl-4,9,16,21-dimetheno-1H,13H-dibenzo[e,k]pyrrolo[3,4-h][1,4,13]oxadiazacyclohexadecene-1,3(2H)-dione, a pharmaceutically acceptable salt, solvate, or ester thereof, or combinations thereof.

8. The method of any one of claims 1-7, wherein administering a therapeutically effective amount of a compound comprising a PKC inhibitor, a GSK3β inhibitor, or an inhibitor of both comprises administering a composition or formulation consisting essentially of a PKC inhibitor, a GSK3β inhibitor, or an inhibitor of both; comprises administering a composition or formulation consisting essentially of a PKC inhibitor; comprises administering a composition or formulation consisting essentially of a GSK3β inhibitor; comprises administering a composition or formulation consisting essentially of an inhibitor of both PKC and GSK3β3; comprises administering a composition or formulation consisting essentially of ruboxistaurin, prodrugs, active metabolites, analogs, or derivatives thereof, a pharmaceutically acceptable salt, solvate, or ester of the foregoing, or combinations thereof; comprises administering a composition or formulation consisting essentially of sotrastaurin, prodrugs, active metabolites, analogs, or derivatives thereof, a pharmaceutically acceptable salt, solvate, or ester of the foregoing, or combinations thereof; comprises administering a composition or formulation consisting essentially of enzastaurin, prodrugs, active metabolites, analogs, or derivatives thereof, a pharmaceutically acceptable salt, solvate, or ester of the foregoing, or combinations thereof; comprises administering a composition or formulation consisting essentially of LY2090314, prodrugs, active metabolites, analogs, or derivatives thereof, a pharmaceutically acceptable salt, solvate, or ester of the foregoing, or combinations thereof; comprises administering a composition or formulation consisting essentially of AZD1080, prodrugs, active metabolites, analogs, or derivatives thereof, a pharmaceutically acceptable salt, solvate, or ester of the foregoing, or combinations thereof; or comprises administering a composition or formulation consisting essentially of AZD2858, prodrugs, active metabolites, analogs, or derivatives thereof, a pharmaceutically acceptable salt, solvate, or ester of the foregoing, or combinations thereof.

9. The method of any one of claims 1-8, wherein administering comprises orally administering.

10. The method of any one of claims 1-9, wherein administering comprises parenterally administering.

11. The method of any one of claims 1-10, wherein the method comprises treating sphingolipidoses or symptoms thereof.

12. The method of any one of claims 1-11, wherein the method comprises treating gangliosidoses or symptoms thereof.

13. The method of any one of claims 1-12, wherein the method comprises treating GM2 gangliosidoses or symptoms thereof.

14. The method of any one of claims 1-13, wherein the method comprises treating Tay-Sachs disease or symptoms thereof.

15. The method of any one of claims 1-13, wherein the method comprises treating Sandhoff disease or symptoms thereof.

16. The method of any one of claims 1-13, wherein the method comprises treating infantile GM2 gangliosidoses or symptoms thereof.

17. The method of any one of claims 1—Error! Reference source not found, wherein the subject is 36 months old or less at the time of starting treatment.

18. The method of any one of claims 1-17, wherein the symptoms include one or more of: progressive loss of motor skills; retinal abnormalities; myoclonic seizures; increased startle response; severe constipation; loss of visceral organ function; cherry red spot; hypotonia; dyskinesia; and dystonia.

19. A method of treating diseases and symptoms associated with mutation in a hexosaminidase subunit alpha (HEXA) gene of a subject, the method comprising administering a therapeutically effective amount of a compound comprising a PKC inhibitor, a GSK3β inhibitor, or an inhibitor of both to a subject in need of treatment.

20. The method of claim 19, wherein the compound comprises ruboxistaurin, sotrastaurin, enzastaurin, LY2090314, AZD1080, AZD2858, prodrugs, active metabolites, analogs, or derivatives of the foregoing or a pharmaceutically acceptable salt, solvate, or ester of the foregoing.

21. The method of claim 19 or claim 20, wherein the method comprises treating Tay-Sachs disease or symptoms thereof.

22. A method of treating diseases and symptoms associated with mutation in a hexosaminidase subunit beta (HEXB) gene of a subject, the method comprising administering a therapeutically effective amount of a compound comprising a PKC inhibitor, a GSK3β inhibitor, or an inhibitor of both to a subject in need of treatment.

23. The method of claim 22, wherein the compound comprises ruboxistaurin, sotrastaurin, enzastaurin, LY2090314, AZD1080, AZD2858, prodrugs, active metabolites, analogs, or derivatives of the foregoing or a pharmaceutically acceptable salt, solvate, or ester of the foregoing.

24. The method of claim 22 or claim 23, wherein the method comprises treating Sandhoff disease or symptoms thereof.

25. The method of any one of claims 19-24, wherein the subject is a mammal.

26. The method of any one of claims 19-25, wherein administering a therapeutically effective amount of a compound comprising a PKC inhibitor, a GSK3β inhibitor, or an inhibitor of both comprises administering a composition or formulation consisting essentially of a PKC inhibitor, a GSK3β inhibitor, or an inhibitor of both; comprises administering a composition or formulation consisting essentially of a PKC inhibitor; comprises administering a composition or formulation consisting essentially of a GSK3β inhibitor; comprises administering a composition or formulation consisting essentially of an inhibitor of both PKC and GSK3β; comprises administering a composition or formulation consisting essentially of ruboxistaurin, prodrugs, active metabolites, analogs, or derivatives thereof, a pharmaceutically acceptable salt, solvate, or ester of the foregoing, or combinations thereof; comprises administering a composition or formulation consisting essentially of sotrastaurin, prodrugs, active metabolites, analogs, or derivatives thereof, a pharmaceutically acceptable salt, solvate, or ester of the foregoing, or combinations thereof; comprises administering a composition or formulation consisting essentially of enzastaurin, prodrugs, active metabolites, analogs, or derivatives thereof, a pharmaceutically acceptable salt, solvate, or ester of the foregoing, or combinations thereof; comprises administering a composition or formulation consisting essentially of LY2090314, prodrugs, active metabolites, analogs, or derivatives thereof, a pharmaceutically acceptable salt, solvate, or ester of the foregoing, or combinations thereof; comprises administering a composition or formulation consisting essentially of AZD1080, prodrugs, active metabolites, analogs, or derivatives thereof, a pharmaceutically acceptable salt, solvate, or ester of the foregoing, or combinations thereof; or comprises administering a composition or formulation consisting essentially of AZD2858, prodrugs, active metabolites, analogs, or derivatives thereof, a pharmaceutically acceptable salt, solvate, or ester of the foregoing, or combinations thereof.

27. A method of treating cells with increased ganglioside accumulation, the method comprising administering an effective amount of a compound comprising a PKC inhibitor, a GSK3β inhibitor, or an inhibitor of both to the cells.

28. The method of claim 27, wherein the cells are in a mammal.

29. The method of claim 28, further comprising identifying the mammal as having cells with ganglioside accumulation.

30. The method of any one of claims 27-29, further comprising measuring GM2 fluorescence of the cells.

31. The method of any one of claims 27-30, wherein administering an effective amount of a compound comprising a PKC inhibitor, a GSK3β inhibitor, or an inhibitor of both comprises administering a composition or formulation consisting essentially of a PKC inhibitor, a GSK3β inhibitor, or an inhibitor of both; comprises administering a composition or formulation consisting essentially of a PKC inhibitor; comprises administering a composition or formulation consisting essentially of a GSK3β inhibitor; comprises administering a composition or formulation consisting essentially of an inhibitor of both PKC and GSK3β; comprises administering a composition or formulation consisting essentially of ruboxistaurin, prodrugs, active metabolites, analogs, or derivatives thereof, a pharmaceutically acceptable salt, solvate, or ester of the foregoing, or combinations thereof; comprises administering a composition or formulation consisting essentially of sotrastaurin, prodrugs, active metabolites, analogs, or derivatives thereof, a pharmaceutically acceptable salt, solvate, or ester of the foregoing, or combinations thereof; comprises administering a composition or formulation consisting essentially of enzastaurin, prodrugs, active metabolites, analogs, or derivatives thereof, a pharmaceutically acceptable salt, solvate, or ester of the foregoing, or combinations thereof; comprises administering a composition or formulation consisting essentially of LY2090314, prodrugs, active metabolites, analogs, or derivatives thereof, a pharmaceutically acceptable salt, solvate, or ester of the foregoing, or combinations thereof; comprises administering a composition or formulation consisting essentially of AZD1080, prodrugs, active metabolites, analogs, or derivatives thereof, a pharmaceutically acceptable salt, solvate, or ester of the foregoing, or combinations thereof; or comprises administering a composition or formulation consisting essentially of AZD2858, prodrugs, active metabolites, analogs, or derivatives thereof, a pharmaceutically acceptable salt, solvate, or ester of the foregoing, or combinations thereof.

32. The method of any one of claims 27-31, wherein administering comprises orally administering or parenterally administering.

33. The method of any one of claims 19-32, comprising administering ruboxistaurin hydrochloride, sulfate, mesylate, succinate, tartrate, acetate, or phosphate.

34. The method of any one of claims 19-32, comprising administering ruboxistaurin mesylate monohydrate.

35. The method of any one of claims 19-32, comprising administering N-desmethyl ruboxistaurin, a pharmaceutically acceptable salt, solvate, or ester thereof, or combinations thereof.

36. The method of any one of claims 19-32, comprising administering (S)-10,11,14,15-tetrahydro-13-hydroxymethyl-4,9,16,21-dimetheno-1H,13H-dibenzo[e,k]pyrrolo[3,4-h][1,4,13]oxadiazacyclohexadecene-1,3(2H)-dione, a pharmaceutically acceptable salt, solvate, or ester thereof, or combinations thereof.

37. A method of identifying a patient with ganglioside accumulation, the method comprising obtaining cells from a patient and measuring GM2 fluorescence of the cells.

38. The method of claim 37, further comprising administering an effective amount of a compound comprising a PKC inhibitor, a GSK3β inhibitor, or an inhibitor of both to a subject in need of treatment.

39. The method of claim 37 or of claim 38, wherein the compound comprises ruboxistaurin, sotrastaurin, enzastaurin, LY2090314, AZD1080, AZD2858, prodrugs, active metabolites, analogs, or derivatives of the foregoing or a pharmaceutically acceptable salt, solvate, or ester of the foregoing.

40. A composition for use in treating lysosomal storage diseases and symptoms thereof, the composition comprising a therapeutically effective amount of a compound comprising a PKC inhibitor, a GSK3β inhibitor, or an inhibitor of both.

41. The composition of claim 40, wherein the compound comprises ruboxistaurin, sotrastaurin, enzastaurin, LY2090314, AZD1080, AZD2858, prodrugs, active metabolites, analogs, or derivatives of the foregoing or a pharmaceutically acceptable salt, solvate, or ester of the foregoing.

42. The composition of claim 40, wherein the compound comprises ruboxistaurin, prodrugs, active metabolites, analogs, or derivatives of the foregoing or a pharmaceutically acceptable salt, solvate, or ester of the foregoing.

43. The composition of claim 40, wherein the composition comprises ruboxistaurin hydrochloride, sulfate, mesylate, succinate, tartrate, acetate, or phosphate.

44. The composition of claim 40, wherein the composition comprises ruboxistaurin mesylate monohydrate.

45. The composition of claim 40, wherein the composition comprises N-desmethyl ruboxistaurin, a pharmaceutically acceptable salt, solvate, or ester thereof, or combinations thereof.

46. The composition of claim 40, wherein the composition comprises (S)-10,11,14,15-tetrahydro-13-hydroxymethyl-4,9,16,21-dimetheno-1H,13H-dibenzo[e,k]pyrrolo[3,4-h][1,4,13]oxadiazacyclohexadecene-1,3(2H)-dione, a pharmaceutically acceptable salt, solvate, or ester thereof, or combinations thereof.

47. The composition of claim 40, wherein the composition consists essentially of a PKC inhibitor, a GSK3β inhibitor, or an inhibitor of both; consists essentially of a PKC inhibitor; consists essentially of a GSK3β inhibitor; consists essentially of an inhibitor of both PKC and GSK3β; consists essentially of ruboxistaurin, prodrugs, active metabolites, analogs, or derivatives thereof, a pharmaceutically acceptable salt, solvate, or ester of the foregoing, or combinations thereof; consists essentially of sotrastaurin, prodrugs, active metabolites, analogs, or derivatives thereof, a pharmaceutically acceptable salt, solvate, or ester of the foregoing, or combinations thereof; consists essentially of enzastaurin, prodrugs, active metabolites, analogs, or derivatives thereof, a pharmaceutically acceptable salt, solvate, or ester of the foregoing, or combinations thereof; consists essentially of LY2090314, prodrugs, active metabolites, analogs, or derivatives thereof, a pharmaceutically acceptable salt, solvate, or ester of the foregoing, or combinations thereof; consists essentially of AZD1080, prodrugs, active metabolites, analogs, or derivatives thereof, a pharmaceutically acceptable salt, solvate, or ester of the foregoing, or combinations thereof; or consists essentially of AZD2858, prodrugs, active metabolites, analogs, or derivatives thereof, a pharmaceutically acceptable salt, solvate, or ester of the foregoing, or combinations thereof.

48. The composition of any one of claims 40-46, wherein the composition is formulated for oral administration.

49. The composition of any one of claims 40-46, wherein the composition is formulated for parenteral administration.

50. The composition of any one of claims 40-49, wherein the disease comprises sphingolipidoses or symptoms thereof.

51. The composition of any one of claims 40-50, wherein the disease comprises gangliosidoses or symptoms thereof.

52. The composition of any one of claims 40-51, wherein the disease comprises GM2 gangliosidoses or symptoms thereof.

53. The composition of any one of claims 40-52, wherein the disease comprises Tay-Sachs disease or symptoms thereof.

54. The composition of any one of claims 40-53, wherein the disease comprises Sandhoff disease or symptoms thereof.

55. The composition of any one of claims 40-54, wherein the disease comprises infantile GM2 gangliosidoses or symptoms thereof.

56. The composition of any one of claims 40-55, wherein the symptoms include one or more of: progressive loss of motor skills; retinal abnormalities; myoclonic seizures; increased startle response; severe constipation; loss of visceral organ function; cherry red spot; hypotonia; dyskinesia; and dystonia.

57. A method of manufacturing a composition for use in treating lysosomal storage diseases and symptoms thereof, the composition comprising a therapeutically effective amount of a compound comprising a PKC inhibitor, a GSK3β inhibitor, or an inhibitor of both.

58. The method of claim 57, wherein the compound comprises ruboxistaurin, sotrastaurin, enzastaurin, LY2090314, AZD1080, AZD2858, prodrugs, active metabolites, analogs, or derivatives of the foregoing or a pharmaceutically acceptable salt, solvate, or ester of the foregoing.

59. The method of claim 57, wherein the compound comprises ruboxistaurin, prodrugs, active metabolites, analogs, or derivatives of the foregoing or a pharmaceutically acceptable salt, solvate, or ester of the foregoing.

60. The method of claim 57, wherein the composition comprises ruboxistaurin hydrochloride, sulfate, mesylate, succinate, tartrate, acetate, or phosphate.

61. The method of claim 57, wherein the composition comprises ruboxistaurin mesylate monohydrate.

62. The method of claim 57, wherein the composition comprises N-desmethyl ruboxistaurin, a pharmaceutically acceptable salt, solvate, or ester thereof, or combinations thereof.

63. The method of claim 57, wherein the composition comprises (S)-10,11,14,15-tetrahydro-13-hydroxymethyl-4,9,16,21-dimetheno-1H,13H-dibenzo[e,k]pyrrolo[3,4-h][1,4,13]oxadiazacyclohexadecene-1,3(2H)-dione, a pharmaceutically acceptable salt, solvate, or ester thereof, or combinations thereof.

64. The method of claim 57, wherein the composition consists essentially of a PKC inhibitor, a GSK3β inhibitor, or an inhibitor of both; consists essentially of a PKC inhibitor; consists essentially of a GSK3β inhibitor; consists essentially of an inhibitor of both PKC and GSK3β; consists essentially of ruboxistaurin, prodrugs, active metabolites, analogs, or derivatives thereof, a pharmaceutically acceptable salt, solvate, or ester of the foregoing, or combinations thereof; consists essentially of sotrastaurin, prodrugs, active metabolites, analogs, or derivatives thereof, a pharmaceutically acceptable salt, solvate, or ester of the foregoing, or combinations thereof; consists essentially of enzastaurin, prodrugs, active metabolites, analogs, or derivatives thereof, a pharmaceutically acceptable salt, solvate, or ester of the foregoing, or combinations thereof; consists essentially of LY2090314, prodrugs, active metabolites, analogs, or derivatives thereof, a pharmaceutically acceptable salt, solvate, or ester of the foregoing, or combinations thereof; consists essentially of AZD1080, prodrugs, active metabolites, analogs, or derivatives thereof, a pharmaceutically acceptable salt, solvate, or ester of the foregoing, or combinations thereof; or consists essentially of AZD2858, prodrugs, active metabolites, analogs, or derivatives thereof, a pharmaceutically acceptable salt, solvate, or ester of the foregoing, or combinations thereof.

65. The method of any one of claims 57-64, wherein the composition is formulated for oral administration.

66. The method of any one of claims 57-64, wherein the composition is formulated for parenteral administration.

67. The method of any one of claims 57-66, wherein the disease comprises sphingolipidoses or symptoms thereof.

68. The method of any one of claims 57-67, wherein the disease comprises gangliosidoses or symptoms thereof.

69. The method of any one of claims 57-68, wherein the disease comprises GM2 gangliosidoses or symptoms thereof.

70. The method of any one of claims 57-69, wherein the disease comprises Tay-Sachs disease or symptoms thereof.

71. The method of any one of claims 57-69, wherein the disease comprises Sandhoff disease or symptoms thereof.

72. The method of any one of claims 57-69, wherein the disease comprises infantile GM2 gangliosidoses or symptoms thereof.

73. The method of any one of claims 57-72, wherein the symptoms include one or more of: progressive loss of motor skills; retinal abnormalities; myoclonic seizures; increased startle response; severe constipation; loss of visceral organ function; cherry red spot; hypotonia; dyskinesia; and dystonia.
